(19) United States
(12) Patent Application Publication
(10) Pub. No.: US 2013/0065910 A1
(43) Pub. Date: Mar. 14, 2013

(54) ORAL MECLIZINE AQUEOUS FORMULATIONS WITH TASTE FLAVORING AGENT

(71) Applicants: Benjamin Tak Kwong Lee, Shatin (HK); Shuai Qian, Shatin (CN); Yan Feng Wang, Shatin (CN)

(72) Inventors: Benjamin Tak Kwong Lee, Shatin (HK); Shuai Qian, Shatin (CN); Yan Feng Wang, Shatin (CN)

(73) Assignee: COMPREHENSIVE DRUG ENTERPRISES LTD., Shatin, NT (HK)

(21) Appl. No.: 13/700,152

(22) PCT Filed: Jun. 2, 2011
(86) PCT No.: PCT/CN2011/075192

§ 371 Date: Nov. 27, 2012

(30) Foreign Application Priority Data
Jun. 4, 2010 (HK) 10105516.2

(51) Int. Cl. A61K 31/495; A61P 1/08

(52) U.S. Cl. 514/255.04

(57) ABSTRACT

An oral meclizine aqueous formulation is disclosed. The oral meclizine aqueous formulation comprises a solubizier for enhancing aqueous solubility of meclizine, and a taster flavoring agent selected from the group consisting of sugar, sucrose, sorbitol, sucralose, saccharin sodium, sodium cyclamate, aspartame, neotame, acesulfame potassium, stevioside, and their mixtures. Preferably the solubilizer is hydroxypropyl-β-cyclodextrin or polyethylene glycol 660 13-hydroxystearate. The oral meclizine aqueous formulation can provide a rapid onset of therapeutic effects of meclizine.

### FIELD OF THE INVENTION

This invention relates to oral meclizine aqueous formulations for preventing and/or treating symptoms relating to motion sickness including nausea, vomiting and dizziness. The formulations are particularly those that include a solubilizer for enhancing aqueous solubility of meclizine and a taste flavoring agent.

### BACKGROUND OF THE INVENTION

Inhibition of nausea and/or emesis in mammals can be induced by stimulation of either the chemoreceptor trigger zone or the emesis (or vomiting) center in central nervous system (CNS). Such stimulation can be caused by afferent stimulation (e.g., tactile pharyngeal impulses, labrynthine disturbances, motion, increased intracranial pressure, pain, distention of viscera or psuchologic factors) or blood borne emritc substances (e.g., as seen during pregnancy, cancer chemotherapy, uremia, radiation therapy, electrolyte and endocrine disturbances, or the presence of chemical emetic substances). Meclizine is known to be a drug for inhibition of nausea and/or emesis in mammals.

It is known that conventional meclizine tablets or capsules have a relatively slow onset of pharmacological activity. For motion sickness, such tablet or capsule should be taken at least 1-2 hours prior to travel in order to ensure absorption of the drug. On the other hand, it is difficult to treat the symptoms once emesis or nausea is triggered.

### OBJECTS OF THE INVENTION

Therefore, it is an object of this invention to resolve at least one or more problems as set forth in the prior art. Particularly, it is an object of the current invention to provide formulations and/or methods for oral administration of meclizine that could be administered with relatively rapid onset of pharmacological effect for preventing and/or treating symptoms relating to motion sickness including nausea, vomiting and dizziness. It is another object of this invention to provide easy-to-use, simple and non-invasive formulations for preventing and/or treating symptoms relating to motion sickness including nausea, vomiting and dizziness. As a minimum, it is an object of this invention to provide the public with a useful choice.

### SUMMARY OF THE INVENTION

Accordingly, this invention provides an oral meclizine aqueous formulation, which includes a solubilizer for enhancing aqueous solubility of meclizine. This formulation further includes a taste flavoring agent selected from the group consisting of sugar, sucrose, sorbitol, sucralose, saccharin sodium, sodium cyclamate, aspartame, neotame, acesulfame potassium, stevioside, and any of their mixtures or combinations.

Preferably, the meclizine is in the form of meclizine hydrochloride, and has a concentration of 0.1 to 40 mg/mL, more preferably 1 to 25 mg/mL, and even more preferably 3 to 10 mg/mL.

Preferably, the taste flavoring agent is selected from the group consisting of sucrose, sucralose, sodium cyclamate and neotame. It is particularly preferred that the taste flavoring agent is sodium cyclamate, and has a concentration of 0.05 to 4% (w/v), more preferably 0.15 to 2% (w/v), and most preferably 0.2 to 1% (w/v). Alternatively, the taste flavoring agent can be scuralose, and has a concentration of 0.05 to 2% (w/v), more preferably 0.15 to 2% (w/v), and even more preferably 0.2 to 1% (w/v).

Advantageously, the solubilizer is hydroxyproply-β-cyclodextrin, and has a concentration of 0.6 to 40% (w/v), more preferably 2 to 30% (w/v), and even more preferably 5 to 20% (w/v). Alternatively, the solubilizer can be polyethylene glycol 660 12-hydroxystearate (Solutol® HS15), and has a concentration of 0.1 to 30% (w/v), more preferably 1 to 20% (w/v), and even more preferably 2.5 to 10% (w/v).

Preferably, the oral meclizine aqueous formulations of the current invention have a dosage volume of 0.5-30 ml, more preferably 2-15 ml and even more preferably 3-10 ml. More preferably, the oral meclizine aqueous formulation of is in a unit-dose packaging for single use.

It is another aspect of the current invention to provide a method of manufacturing an oral meclizine aqueous formulation including the steps of mixing meclizine and water with a solubilizer for enhancing aqueous solubility of meclizine and a taste flavoring agent selected from the group consisting of sugar, sucrose, sorbitol, sucralose, saccharin sodium, sodium cyclamate, aspartame, neotame, acesulfame potassium, stevioside, and their mixtures.

It is yet another aspect of this invention to provide the use of an oral meclizine aqueous formulation for preventing and/or treating symptoms of motion sickness including nausea or emesis, and dizzyness, said oral meclizine aqueous formulation including a solubilizer for enhancing aqueous solubility of meclizine and a taste flavoring agent selected from the group consisting of sugar, sucrose, sorbitol, sucralose, saccharin sodium, sodium cyclamate, aspartame, neotame, acesulfame potassium, stevioside, and their mixtures.

The use of meclizine, a solubilizer for enhancing solubility of meclizine in water and a taste flavoring agent selected from the group consisting of sugar, sucrose, sorbitol, sucralose, saccharin sodium, sodium cyclamate, aspartame, neotame, acesulfame potassium, stevioside, and their mixtures in manufacturing an oral meclizine aqueous formulation for treating nausea or emesis, said oral meclizine aqueous formulation.

### BRIEF DESCRIPTION OF THE DRAWINGS

Preferred embodiments of the present invention will now be explained by way of example and with reference to the accompanying drawings in which:

FIG. 1 shows the solubility of meclizine hydrochloride in solutions containing Solutol® HS15 and HP-β-CD;

FIG. 2 shows the plasma concentration curves following administration of meclizine solution and suspension of meclizine tablet on rats;

FIG. 3 shows the plasma concentration-time profiles of meclizine in 6 beagle dogs following an oral dose 1×Meclizine solution 25 mg of the current invention (test) or 1×Meclizine marketing tablet 25 mg (reference); and

FIG. 4 shows the mean (SD) plasma concentration of 2 oral formulation of meclizine 25 mg (Test, MOS; Reference, MOT) in 19 human subjects.

### DETAILED DESCRIPTION OF THE PREFERRED EMBODIMENTS

This invention is now described by way of example with reference to the figures in the following paragraphs. Objects, features, and aspects of the present invention are disclosed in or are apparent from the following description. It is to be understood by one of ordinary skilled in the art that the present discussion is a description of exemplary embodiments only, and is not intended as limiting the broader aspects of the present invention, which broader aspects are embodied in the exemplary constructions.

Meclizine (molecular weight 390.9) is the common name of 1-[(4-chlorophenyl)-phenyl-methyl]-4-[(3-methylphenyl)methyl]piperazine, which has the following chemical structure:

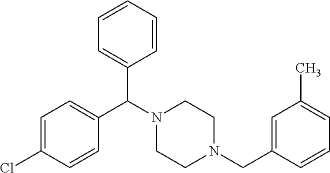 [SMILES: Cc1cccc(CN2CCN(C(c3ccccc3)c3ccc(Cl)cc3)CC2)c1]

Meclizine is a piperazine-derivative antihistamine that is used as an antiemetic. Meclizine hydrochloride occurs as a white or slightly yellowish, crystalline powder with a slight odor, and is practically insoluble in water while slightly soluble in alcohol. Due to this insolubility, existing meclizine tablets or capsules are known to be tasteless.

Meclizine has CNS depressant, anticholinergic, antiemetic, antispasmodic, and local anesthetic effects in addition to antihistaminic activity. The drug depresses labyrinth excitability and conduction in vestibular-cerebellar pathways. The antiemetic and antimotion-sickness actions of meclizine result, at least in part, are from its central anticholinergic and CNS depressant properties.

As an antihistamine, meclizine shows marked protective activity against nebulized histamine and lethal doses of intravenously injected histamine in guinea pigs. It has a marked effect in blocking the vasodepressor response to histamine, but only a slight blocking action against acetylcholine. Its activity is relatively weak in inhibiting the spasmogenic action of histamine on isolated guinea pig ileum.

Meclizine is typically administered therapeutically as the hydrochloride salt. The dose for adult and children 12 years of age and over was reported to be 25 mg to 50 mg in tablet form.

It is known to use solubilizers to dissolve meclizine in water to form aqueous solutions. WO 2009/059120 describes injectable meclizine formulations using various cyclodextrins to dissolve meclizine in water. The purpose of this WO 2009/059120 is to provide meclizine solutions for injection. As injection is considered as a route different from oral administration, there would be no reason to look into WO 2009/059120 for development of oral meclizine formulations.

Indeed, it was found in the present invention that the meclizine solutions of WO 2009/059120 are not suitable for oral administration. Such solutions were found to be too bitter to be orally administered, which is unexpected as meclizine tablets and capsules are tasteless.

Further, it was found in the oral meclizine aqueous formulations of the current invention provides rapid onset of action as compared with existing commercially available tablet. In particular, it has been found that the meclizine formulations of the current reach peak plasma concentration significantly faster than existing meclizine tablets.

The oral meclizine aqueous formulations of the current invention apparently contain meclizine and water. The meclizine in the aqueous formulations of the current invention can be in the form of meclizine hydrochloride, and has a concentration of from 0.1 to 40 mg/ml, more preferably from 1 to 25 mg/ml, most preferably 3 to 10 mg/ml. These specific ranges were chosen so as to maintain the volume of the formulation to be about_{—}0.5-30 ml, more preferably 2-15 ml and even more preferably 3-10 ml, which was found to be convenient to be packed and carried around by the user and easy to be used. Unit-dose packaging for single use for example sache packaging with 5 ml can be prepared with the formulations of the current invention. There is no existing similar unit-dose packaging (single use) for preventing and/or treating symptoms relating to motion sickness including nausea, vomiting and dizziness.

Suitable solubilizer that can be used in the current invention include hydroxyproply-β-cyclodextrin (HP-β-CD) and polyethylene glycol 660 12-hydroxystearate, the later is commonly sold under the brand Solutol® HS15.

If HP-β-CD is used as the solubilizer, then the concentration of HP-β-CD is preferred to be from 0.6 to 40% (w/v), more preferably from 2 to 30% and even more preferably from 5 to 20%. For the case of Solutol® HS15, the concentration is preferred to be from 0.1 to 30% (w/v), more preferably from 1 to 20% and even preferably from 2.5 to 10%. These concentration ranges were chosen to avoid undesirably high viscosity and adverse effect to the taste, while maintaining the solubilizing function of the solubilizers.

The formulations of the present invention may, alternatively, contain some other pharmaceutically acceptable solubilizers to enhance the solubility of meclizine hydrochloride. If desired, minor amount of non-aqueous solvents may be included without affect water being the major solvent of the formulations. Such non-aqueous solvents include ethanol, propylene glycol, polyethylene glycol, benzyl benzoate, polyoxyethylene castor oil derivates.

To improve the taste of the formulations to become acceptable for oral administration, taste flavoring agents are included in the formulations of the current invention. Other than taste, such taste flavoring agents should be pharmaceutically compatible with meclizine, and not affecting the solubility of meclizine in the presence of the solubilizer in water. It was found that some taste flavoring agents are not suitable as they precipitate meclizine out of the formulations. Taste flavoring agents that are suitable to the formulations of the current invention include sugar, sucrose, sorbitol, sucralose, saccharin sodium, sodium cyclamate, aspartame, neotame, acesulfame potassium, stevioside, and their mixtures. The preferred taste flavoring agents are sucrose, sucralose, sodium cyclamate, and neotame; and the more preferred taste flavoring agents are sucralose and sodium cyclamate.

When the taste flavoring agent is sodium cyclamate, the concentration of sodium cyclamate is referred to be 0.05 to 4% (w/v), more preferably 0.15 to 2% (w/v), and even more preferably 0.2 to 1% (w/v). If scuralose is used as the taste flavoring agent, then the concentration is preferred to be 0.05 to 2% (w/v), more preferably 0.15 to 2% (w/v), and even more preferably 0.2 to 1% (w/v). It should be noted that the taste of the oral meclizine aqueous formulations of the current invention would not be improved if too low concentration of the taste flavoring agent is used. The upper limit of the concentration of the taste flavoring agent may be affected by various factors including acceptable daily intake recommended by the authorities.

It is known that meclizine will precipitate from aqueous solutions at high pH even with the presence of solubilizers. Therefore, the oral meclizine aqueous formulations of the current invention prefer to have a pH of from 1 to 5, more preferably from 1.5 to 4.

Other than the above components, preservative like sodium benzoate and pH adjusting agent like hydrochloric acid may present.

Various embodiments of the current invention are described in the following examples, which are for illustrative purpose and shall not be considered as limiting in any manner.

### Example 1

### Solubility of Meclizine Hydrochloride in Solutol® HS15 and HP-β-CD

The ability of Solutol® HS15 and HP-β-CD to enhance the aqueous solubility of meclizine hydrochloride is determined in this example.

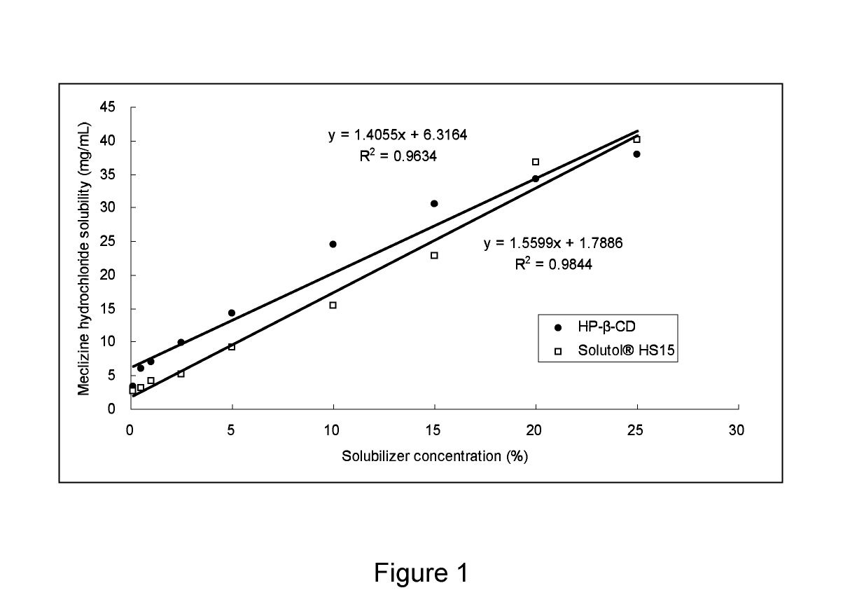

Aqueous solutions containing 0.1, 0.5, 1, 2.5, 5, 10, 15, 20, and 25% (w/v) (w/v) of Solutol® HS15 or HP-β-CD solubilizers were prepared. 10 mg samples of meclizine hydrochloride were added to a 4 ml portion of each of the above solutions. The samples were left to be stirred overnight at room temperature (22° C.). If during this time all drug dissolved, additional meclizine was added until a suspension was formed (i.e. saturated drug solution formed). After further stirring for up to 24 hours, suspensions were centrifuged at 14000 rpm for 10 minutes, and then the supernatants were filtered (0.45 μm filter, Sartorius, Germany) to obtain saturated meclizine hydrochloride solutions. The filtrate samples were diluted and detected by HPLC and the concentration of meclizine hydrochloride in the samples was calculated with the calibration curve made from standard solutions of meclizine hydrochloride. Solubility data are presented in Table 1 below and FIG. 1.

TABLE 1
Solubility of meclizine hydrochloride in Solutol ® HS15 and HP-β-CD
solutions

| | |
|---|---|
| | Meclizine |
| Solubilizer | hydrochloride solubility (mg/ml) |

| Concentration (%) | Solutol ® HS15 | HP-β-CD |
|---|---|---|
| 0.1 | 2.6 | 3.4 |
| 0.5 | 3.2 | 6.1 |
| 1 | 4.3 | 7.1 |
| 2.5 | 5.2 | 9.9 |
| 5 | 9.2 | 14.3 |
| 10 | 15.4 | 24.6 |
| 15 | 22.8 | 30.5 |
| 20 | 36.8 | 34.3 |
| 25 | 40.1 | 37.9 |

The enhancement of meclizine hydrochloride solution by both Solutol® HS15 and HP-β-CD appears to be relatively linear over the concentration range of 0.1-25%.

### Example 2

### Oral Solution Containing 5 mg/ml Meclizine Hydrochloride

The following oral meclizine aqueous formulations were prepared and packaged in conventional oral liquid bottle or plastic bag.

TABLE 2
Examples of oral meclizine hydrochloride aqueous formulations

| | Formula | Mg. per |
|---|---|---|
| | % w/v | Dose (5 ml) |
| FORMULATION 1 | | |
| Meclizine HCl | 1 | 50 |
| Solutol ® HS15 | 15 | 600 |
| Sucrose | 12 | 600 |
| Saccharin sodium | 0.03 | 1.5 |
| Sodium benzoate | 0.18 | 9 |
| Beet red | 0.005 | 0.25 |
| Cherry flavor | 0.00015 | 0.0075 |
| Water High Grade | 71.78 | 4170.24 |
| | 100 | 5431 |
| FORMULATION 2 | | |
| Meclizine HCl | 0.5 | 25 |
| Ethanol | 5 | 250 |
| Propylene glycol | 15 | 750 |
| Solutol ® HS15 | 5 | 250 |
| Sucrose | 8 | 400 |
| Sucralose | 0.2 | 10 |
| Benzoic acid | 0.2 | 10 |
| Amaranth | 0.005 | 0.25 |
| Orange flavor | 0.00015 | 0.0075 |
| Water High Grade | 66.09 | 3654.74 |
| | 100 | 5350 |
| FORMULATION 3 | | |
| Meclizine HCl | 0.5 | 25 |
| HP-β-CD | 8 | 400 |
| Sucrose | 12 | 600 |
| Sodium cyclamate | 1 | 50 |
| Sodium benzoate | 0.18 | 9 |
| Beet red | 0.005 | 0.25 |
| Orange flavor | 0.00015 | 0.0075 |
| Water High Grade | 78.31 | 4321.74 |
| | 100 | 5406 |
| FORMULATION 4 | | |
| Meclizine HCl | 0.5 | 25 |
| Tween 80 | 5 | 250 |
| Sodium cyclamate | 1.4 | 70 |
| Benzoic acid | 0.2 | 10 |
| Beet red | 0.005 | 0.25 |
| Cherry flavor | 0.00015 | 0.0075 |
| Water High Grade | 92.89 | 4729.74 |
| | 100 | 5085 |
| FORMULATION 5 | | |
| Meclizine HCl | 0.5 | 25 |
| Cremophor ® RH40 | 7.5 | 375 |
| Sucrose | 12 | 600 |
| Sucralose | 0.2 | 10 |
| Benzoic acid | 0.2 | 10 |
| Beet red | 0.005 | 0.25 |
| Cherry flavor | 0.00015 | 0.0075 |
| Water High Grade | 79.59 | 4385.74 |
| | 100 | 5406 |

### Example 3

### Pharmacokinetic Performance of Meclizine Formulation on Rats

Four Sprague-Dawley male rats each weight 225 to 250 grams were administered orally by gastric perfusion at a dosage level of 20 mg/kg of body weight of the suspension of existing meclizine tablet and the follow composition:

10 mg/ml of meclizine hydrochloride dissolves in 12% of HP-β-CD solution

Blood samples were collected from tail vein at predetermined time points (0, 5, 10, 15 and 30 min). To 100 μL of plasma sample, an aliquot of 100 μL of 30 ng/mL flunarizine (Internal standard) and 100 μL of acetonitrile was added, which was followed by vortex mixing and centrifugation for 8 min at 14000 rpm to precipitate the protein. The supernatants were transferred to sample vials and 10 μL of aliquots were injected into LC-MS system for analyzing.

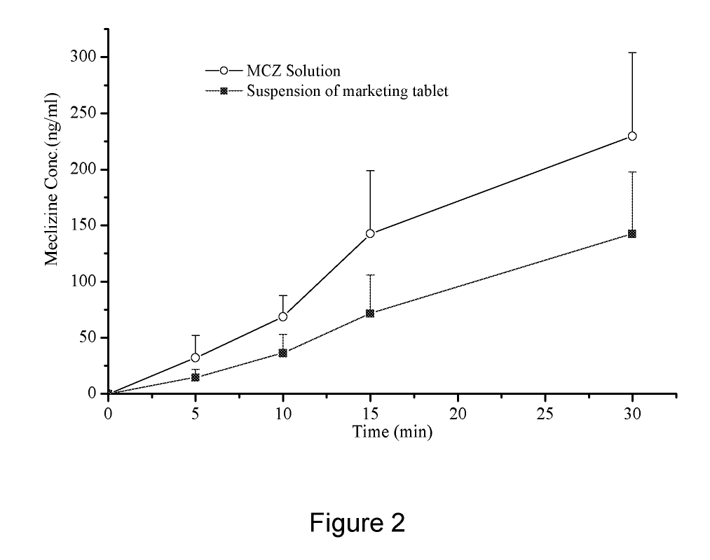

The comparison of plasma-concentration profiles between meclizine solution and suspension of the existing meclizine tablets was shown in FIG. 2.

Rats administered with meclizine solution showed higher plasma concentrations than those given with suspension of meclizine tablet at all of the same time points.

### Example 4

### Pharmacokinetic Performance of Meclizine Formulation on Beagle Dogs

The bioavailability study on 6 Beagle dogs was conducted using a single dose, two-treatment, two-period, two-sequence crossover design. 6 Beagle dogs were all healthy, with mean age of 11.3±0.5 months (range 11-12 months), mean weight of 13.0±0.5 kg (range 12.5-13.7 kg).

Each dog underwent two study sessions, during which they received a single dose of 25 mg meclizine from one of two formulations, 1×Meclizine solution 25 mg (FORMULATION 1 in Table 2, test) or 1×Meclizine marketing tablet 25 mg (reference), and were always followed by forced drinking of 10 ml of water. Venous blood samples were collected at pre-dose (Oh) and then at 0.083, 0.167, 0.25, 0.5, 0.75, 1, 1.5, 2, 3, 4, 6, 8, 12 and 24 h post-dose. Blood samples were collected from a catheter, which was placed in the forelimb vein before dosing. Each time 2 ml of blood was drawn and stored in a sodium heparin tube. All blood samples were centrifuged immediately at 4000 rpm for 10 minutes at room temperature, and separated plasma samples were transferred into polypropylene tubes and stored at −30° C. until assay.

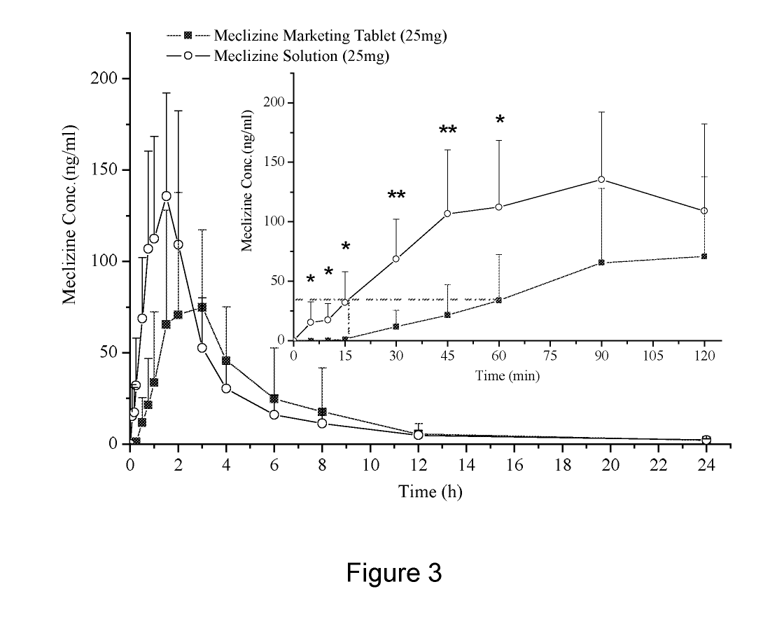

The plasma concentrations were determined by LC/MS system and the plasma-concentration profiles of test and reference formulations was depicted in FIG. 3.

The mean values of the main pharmacokinetic parameters of meclizine were summarized in Table 3 below:

TABLE 3
The mean values of the main pharmacokinetic parameters of meclizine

| | Meclizine tablet | Meclizine solution |
|---|---|---|
| | (reference) | (test) |
| tₘₐₓ (h) | 2.58 ± 0.92 | 1.33 ± 0.58* |
| Cₘₐₓ(ng/ml) | 93.68 ± 49.27 | 158.32 ± 58.12 |
| AUCₗₐₛₜ (ng · h/ml) | 389.72 ± 221.37 | 444.62 ± 142.09 |
| AUCₜₒₜ (ng · h/ml) | 411.56 ± 216.77 | 472.80 ± 143.01 |

*significant difference (p < 0.05)

Statistically significant difference in tₘₐₓ was observed (p=0.046), with the meclizine formulations of the current invention reach tₘₐₓ earlier than the reference by more than one hour. Furthermore, the meclizine plasma concentrations in the first hour of the oral meclzine aqueous formulation of the current invention were found to be significantly higher than those of the reference.

### Example 5

### The Clinical Pharmacokinetic Study on Human

The present study is to investigate the pharmacokinetics of the new formulation and compare the meclizine oral solution of this invention (MOS) to the marketed tablet (25 mg, MOT) in healthy volunteers.

### Method:

The study was conducted using a single dose, two-treatment, two-period, two-sequence crossover design in 20 healthy subjects. Each subject underwent two study sessions, during which they received a single oral dose of 25 mg meclizine from one of the two formulations. Venous blood samples were collected at different time intervals up to 24 h post-dose. Plasma meclizine concentrations were determined by a validated HPLC-MS/MS method. The standard non-compartmental method was used to generate the pharmacokinetic parameters of meclizine, including Cₘₐₓ, Tₘₐₓ, T_{1/2}, AUC₀₋₂₄, AUC_{0-∞}. Analysis of variance (ANOVA) on Cₘₐₓ, AUC₀₋₂₄, AUC_{0-∞} was performed using the General Linear Model (GLM). The 90% confidence intervals (90% CIs) for the mean differences in Cₘₐₓ, AUC₀₋₂₄, AUC_{0-∞} between the two formulations were calculated by two one-sided t-test. Differences in Tₘₐₓ and T_{1/2} were compared between the two formulations, using Wilcoxon signed-rank test and paired Student's t-test, respectively. The mean onset time of the two different dosage forms were also compared.

### Results:

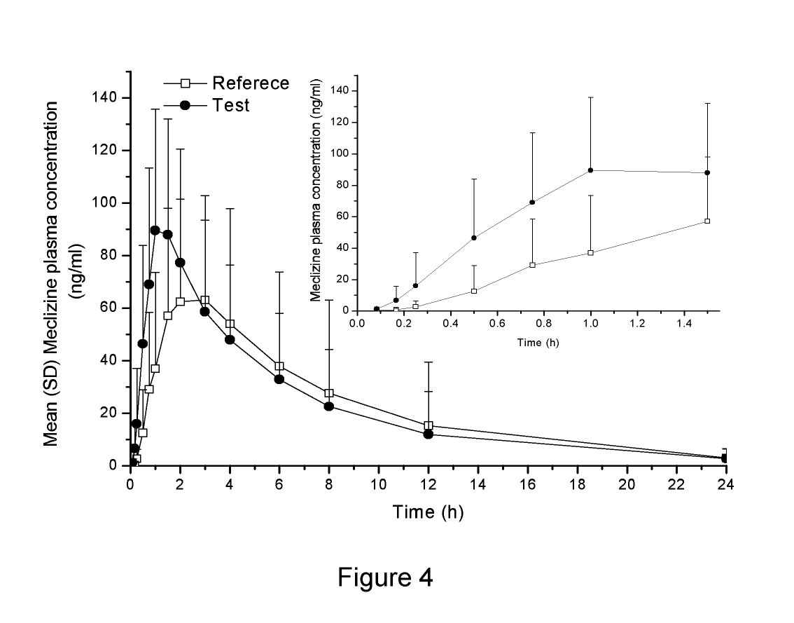

Twenty subjects enrolled in both sessions of the study. However one subject was excluded for consuming excessive caffeinated beverage prior to the study visit and therefore was not included in data analysis due to protocol violation. The mean plasma concentration-time data of meclizine following an oral dose of 1×MOS 25 mg in 5 ml solution (test) or 1×MOT 25 mg tablet (reference) in 19 human subjects are summarized in Table 4., while Table 5. shows the comparative analysis of these pharmacokinetic parameters. FIG. 4 shows the mean plasma concentration-time profiles of meclizine in 19 subjects.

TABLE 4
Pharmacokinetic parameters of oral meclizine 25 mg (test or reference
formulation) after a single dose in healthy adult volunteers (n = 19)
[values are mean (SD)].

| | MOS | MOT |
|---|---|---|
| Parameter | (test) | (reference) |
| Tₘₐₓ (h)* | 1.28 (0.74) | 3.11 (1.35) |
| Cₘₐₓ (ng/ml)* | 99.43 (48.34) | 80.07 (51.85) |
| T_{1/2} (h) | 5.24 (0.82) | 5.21 (0.80) |
| AUC₀₋₂₄ | 542.00 (410.53) | 544.29 (511.62) |
| (ng · h/ml) | | |
| AUC_{0-∞} | 564.03 (439.96) | 566.54 (534.75) |
| (ng · h/ml) | | |

*P < 0.05, significant difference.

TABLE 5
Comparative analysis of pharmacokinetic parameters of MOS (test) and
MOT (reference)

| Parameter | Difference, Mean, h | Difference, 90% CI, h |
|---|---|---|
| Tₘₐₓ | −1.83 | −2.42 to −1.23 |

| | | | Geometric | |
|---|---|---|---|---|
| | Geometric | Geometric mean | mean | |
| Parameter | mean (MOS) | (MOT) | ratio (T/R) | 90% CI |
| Cₘₐₓ | 90.9869 | 68.4219 | 132.98 | 121.22-145.88 |
| AUC₀₋₂₄ | 465.042 | 446.517 | 104.15 | 95.56-112.33 |
| AUC_{0-∞} | 481.4185 | 464.5269 | 103.64 | 96.20-111.65 |

The mean Tₘₐₓ of MOS (test) was 2.43 folds shorter than MOT (1.28 h vs 3.11 h), while the mean Cₘₐₓ was slightly greater (1.24 fold) when compared with MOT. The geometric mean ratios (90% CI) of Cₘₐₓ, AUC₀₋₂₄ and AUC_{0-∞} were 132.98% (121.22-145.88%), 104.15% (95.56-112.33%), and 103.64% (96.20-111.65%) respectively, for MOS 25 mg versus MOT 25 mg indicating no significant difference in bioavailability between the 2 formulations.

### CONCLUSION

As shown in FIG. 4, it can be seen that the meclizine plasma concentration with administration of test formulation is apparently higher that reference formulation in the first 1.0 hour. Taking into account the tolerability of meclizine doses far exceeding the dose studied (up to 225 mg/day for 9.5 years with no adverse effect reported) and the parallel course of plasma drug concentrations with respect to the reference formulation at the elimination phase of the curve, a higher risk due to Cₘₐₓ, dose dumping, or higher redosing is not expected to be necessary.

In view of similar bioavailability but much shorter peak time of the plasma meclizine concentration from the new solution formulation, it has been demonstrated that the new meclizine solution formulation could produce a much quicker onset of action than the existing tablet formulation.

The above results show that the oral meclizine aqueous formulations of this invention provide more rapid onset of therapeutic effects of meclizine than existing products in tablets. Due to this rapid onset effect of the current invention, users can take the formulations of the current invention shortly before traveling, say 20 to 30 minutes, while achieving therapeutic effects of meclizine. This is useful in situations when urgent traveling is required, or the users forget to take the drug two to three hours before traveling. Comparing with injectable solutions, the oral meclizine aqueous formulations of this invention are easier to be administrated and require no medical skills.

To allow the formulations of the current invention to be carried easily to be used whenever necessary, it would be advantageous to have a dosage volume of 0.5-30 ml, more preferably 2-15 ml and even more preferably 3-10 ml for the meclizine aqueous formulations of this invention.

While the preferred embodiment of the present invention has been described in detail by the examples, it is apparent that modifications and adaptations of the present invention will occur to those skilled in the art. Furthermore, the embodiments of the present invention shall not be interpreted to be restricted by the examples or figures only. It is to be expressly understood, however, that such modifications and adaptations are within the scope of the present invention, as set forth in the following claims. For instance, features illustrated or described as part of one embodiment can be used on another embodiment to yield a still further embodiment. Thus, it is intended that the present invention cover such modifications and variations as come within the scope of the claims and their equivalents.

1. An oral meclizine aqueous formulation including:
a solubilizer for enhancing aqueous solubility of meclizine; and
a taste flavoring agent selected from the group consisting of sugar, sucrose, sorbitol, sucralose, saccharin sodium, sodium cyclamate, aspartame, neotame, acesulfame potassium, stevioside, and their mixtures.

2. The oral meclizine aqueous formulation of claim 1, wherein the meclizine is in the form of meclizine hydrochloride, and has a concentration of 0.1 to 40 mg/mL.

3. The oral meclizine aqueous formulation of claim 2, wherein the meclizine has a concentration of 1 to 25 mg/mL.

4. The oral meclizine aqueous formulation of claim 3, wherein the meclizine has a concentration of 3 to 10 mg/mL.

5. The oral meclizine aqueous formulation of claim 1, wherein the taste flavoring agent is selected from the group consisting of sucrose, sucralose, sodium cyclamate and neotame.

6. The oral meclizine aqueous formulation of claim 5, wherein the taste flavoring agent is sodium cyclamate, and has a concentration of 0.05 to 4% (w/v).

7. The oral meclizine aqueous formulation of claim 6, wherein the taste flavoring agent has a concentration of 0.15 to 2% (w/v).

8. The oral meclizine aqueous formulation of claim 7, wherein the taste flavoring agent has a concentration of 0.2 to 1% (w/v).

9. The oral meclizine aqueous formulation of claim 5, wherein the taste flavoring agent is scuralose has a concentration of 0.05 to 2% (w/v).

10. The oral meclizine aqueous formulation of claim 9, wherein the taste flavoring agent has a concentration of 0.15 to 2% (w/v).

11. The oral meclizine aqueous formulation of claim 10, wherein the taste flavoring agent has a concentration of 0.2 to 1% (w/v).

12. The oral meclizine aqueous formulation of claim 1, wherein the solubilizer is hydroxyproply-β-cyclodextrin, and has a concentration of 0.6 to 40% (w/v).

13. The oral meclizine aqueous formulation of claim 12, wherein the solubilizer has a concentration of 2 to 30% (w/v).

14. The oral meclizine aqueous formulation of claim 13, wherein the solubilizer has a concentration of 5 to 20% (w/v).

15. The oral meclizine aqueous formulation of claim 1, wherein the solubilizer is polyethylene glyco 660 12-hydroxystearate, and has a concentration of 0.1 to 30% (w/v).

16. The oral meclizine aqueous formulation of claim 15, wherein the solubilizer has a concentration of 1 to 20% (w/v).

17. The oral meclizine aqueous formulation of claim 16, wherein the solubilizer has a concentration of 2.5 to 10% (w/v).

18. The oral meclizine aqueous formulation of any one of claims 1 to 17 having a dosage volume of 0.5 to 30 ml.

19. The oral meclizine aqueous formulation of claim 18 having a dosage volume of 2 to 15 ml.

20. The oral meclizine aqueous formulation of claim 19 having a dosage volume of 3 to 10 ml.

21. The oral meclizine aqueous formulation of any one of claims 18 to 20 in a unit-dose packaging for single use.

22. A method of manufacturing an oral meclizine aqueous formulation including the steps of mixing meclizine and water with a solubilizer for enhancing solubility of meclizine in water and a taste flavoring agent selected from the group consisting of sugar, sucrose, sorbitol, sucralose, saccharin sodium, sodium cyclamate, aspartame, neotame, acesulfame potassium, stevioside, and their mixtures.

23. The use of an oral meclizine aqueous formulation for preventing and/or treating symptoms of motion sickness including nausea or emesis, and dizziness, said oral meclizine aqueous formulation including a solubilizer for enhancing solubility of meclizine in water and a taste flavoring agent selected from the group consisting of sugar, sucrose, sorbitol, sucralose, saccharin sodium, sodium cyclamate, aspartame, neotame, acesulfame potassium, stevioside, and their mixtures.
